(19) United States
(12) Patent Application Publication
(10) Pub. No.: US 2002/0010471 A1
(43) Pub. Date: Jan. 24, 2002

(54) Methods for injecting materials into bone

(76) Inventors: John F. Wironen, Alachua, FL (US); Harvinder Sandhu, Scarsdale, NY (US); Tom Brewer, Alachua, FL (US)

Correspondence Address: Timothy H. Van Dyke, Bencen & Van Dyke, P.A., 1630 Hillcrest Street, Orlando, FL, 32803, US

(21) Appl. No.: 09/776,605

(22) Filed: Feb. 2, 2001

Related U.S. Application Data
(60) Provisional application No. 60/180,456, filed on Feb. 4, 2000.

(51) Int. Cl. A61F 2/00; A61F 2/32; A61F 2/34; A61F 2/36; A61F 2/38; A61B 17/58; A61B 17/60

(52) U.S. Cl. 606/92

(57) ABSTRACT

Disclosed herein are novel devices, kits, and methods for an improved system of injecting materials into bone and other tissues. Specifically exemplified herein is an assembled trocar and catheter device for insertion and attachment to a site of need, wherein the trocar is removable and wherein the catheter comprises an end for attaching to a syringe or similar device containing biomaterials to be injected into the site of need.

### BACKGROUND OF THE INVENTION

Osteoporosis is a potentially debilitating disease that involves the decalcification and increased brittleness of bones. While both men and women can be affected by the disease, the greatest incidence occurs in postmenopausal women. One commonly occurring manifestation of osteoporosis is a hunching over caused by a decrease in height and mass of the vertebrae, specifically the vertebral body. This symptom can create substantial pain, and can result in life threatening pain and gastrointestinal complications for osteoporosis sufferers. One surgical technique that has been developed to treat this symptom of osteoporosis is vertebralplasty, the intention of which is to restore height and mass to the vertebral body through injection of a polymethylmethacrylate (PMMA) bone cement. Society For Biomaterials, 25^{th} Annual Meeting, “Osteoporosis: A Biomechanical Study of Polymethylmethacrlate Use In Vertebral Bodies” (1999). However, current vertebralplasty techniques are not without drawbacks. First, PMMA tends to squirt out of the vertebral body; since PMMA produces a strong exothermic reaction, the PMMA that squirts out may cause damage to surrounding tissues. Second, PMMA does not degrade and may increase or worsen resorption of the remainder of the vertebra. Third, the PMMA may serve as a nidus for infection. Fourth, since PMMA does not degrade, it obstructs the underlying and surrounding tissues from observation with X-rays or MRI.

### SUMMARY OF THE INVENTION

The subject invention pertains to a novel method and device for performing vertebralplasty. According to one aspect, the subject invention pertains to a method of treating osteoporotic degradation of a bone comprising the steps of engaging a catheter device into said bone, said catheter device being elongated and comprising an attachment means disposed thereon for attaching an injection device containing a filler; and injecting said filler into said bone.

According to another aspect, the subject invention pertains to a kit for repairing osteoporotic bone comprising a catheter device, said catheter being elongated and comprising a first end having threads disposed thereon and a second end having an attachment means disposed thereon; and a syringe containing a filler.

A further aspect of the subject invention pertains to a device for injecting materials into bone comprising a threaded catheter and an internal removable trocar.

These and other advantageous aspects of the subject invention are further described below.

### DESCRIPTION OF THE DRAWINGS

FIG. 1 shows a side view of an embodiment of the subject invention that comprises a catheter and trocar which are brought together to form a trocar/catheter assembly.

FIG. 2 shows a side view of another embodiment of the subject invention that comprises a catheter having a threaded, trocar end.

### DESCRIPTION OF THE PREFERRED EMBODIMENTS

The subject invention pertains to devices, kits and methods for infusing materials into a bone. According to one embodiment, the subject invention is directed to a method of treating osteoporotic degradation of a bone comprising the steps of engaging a catheter device into said bone, said catheter device being elongated and comprising an attachment means disposed thereon for attaching an injection device containing a filler; and injecting said filler into said bone.

In another embodiment, the subject invention is directed to a kit for repairing osteoporotic bone comprising a catheter device, said catheter being elongated and comprising a first end having threads disposed thereon and a second end having an attachment means disposed thereon; and a syringe containing a filler.

In a further embodiment, the subject invention is directed to a device for injecting materials into bone comprising a threaded catheter and an internal removable trocar. The subject device may also have disposed on one end an attachment means, e.g., Luer-lock fitting, for attaching a syringe, whereby a syringe of any filler can then be attached to the luer-lock fitting and the filler material can then be squirted through the catheter and into the marrow cavity. One filler that may be used is a composition comprising mineralized particles (e.g., corticocancellous chips or “CCC” of a size from about 100 to 1000 microns, more preferably 500 to 850 microns), ground bone powder (preferably from about of 100 to 1000 microns, more preferably 500 to 850 microns), a biactive ceramic such as a non-degradable or degradable hydroxyapatite, bioactive glass, and the like, osteogenic paste, chondrogenic paste, carrier associated Growth Factors, carrier associated mineralized particles, morsellized skin or other tissue, Fibrin powder, Fibrin/plasminogen glue, Demineralized Bone Matrix (DBM)/glycerol, DBM/pleuronic F127, DBM/CCC/F127, polyesters, polyhydroxy, compounds, polyvinyl compounds, polyamino compounds, polycarbonate compounds, and mixtures of one or more of these compositions. According to a preferred aspect, the filler comprises a non-degradable hydroxyapatite obtained by calcining bone apatite obtained by neutralizing the acid and then heating the resulting powder to 1400 degrees Celcius in a reducing atmosphere followed by a slow cooling to effect annealing. The non-degradable hydroxyapatite would then be mixed with demineralized bone matrix in order to obtain an implant material that was effectively 90-100% hydroxyapatite particles larger than 500 microns but smaller than 1 mm. The voids between particles could be filled with a bone paste such as disclosed, for example, in WO98/40113.

The subject methods and materials provide a way of performing vertebralplastics or other bone injection surgeries in a minimally invasive manner. Additionally, the nature of the catheter device allows the pressurization of the injected material to restore vertebral body height without the use of the balloon technique such as that disclosed in U.S. Pat. No. 4,969,888. Catheter devices useful in accord with the teachings herein include, but are not limited to, the devices outlined in U.S. Pat. Nos. 5,601,559; 5,192,282; 4,366,822; and 4,258,722.

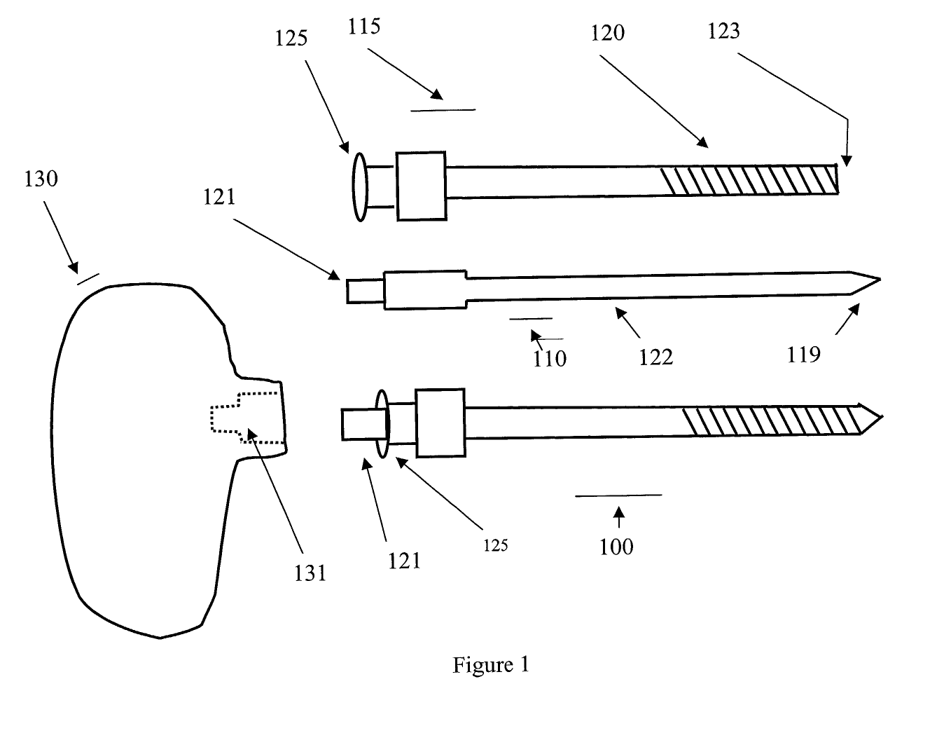

Turning to FIG. 1, an embodiment 100 is shown which is directed to an elongated, hollow device which comprises a sharp trocar 110 and a threaded catheter 115 with threads 120 disposed proximate to an open end 123, wherein the trocar 110 can be removably inserted into the catheter 115 to form a trocar/catheter assembly 117. The trocar 110 facilitates insertion of the catheter 115 through the skin and to the site of need. Preferably, the trocar/catheter assembly is plunged and screwed into the cortex of a vertebral body. Other examples of bones which may treated in accord with the teachings herein include, but are not limited to, the clavicle, femur, humerus, hip, and scapula. Upon insertion of the trocar/catheter assembly 117 into the site of need, the trocar 110 is preferably removed, thereby leaving the catheter secured in place.

The catheter comprises a luer-lok end 125 for attaching a syringe or other device containing a biomaterial to be injected into the site of need. As shown, the trocar 110 comprises a sharp end 119, a peg end 121, and an elongated body portion 122. The peg end 121 is designed to be engaged to a handle 130 at a receiving socket 131, whereby manipulation of the trocar/assembly is facilitated upon engagement to the trocar/catheter assembly 117.

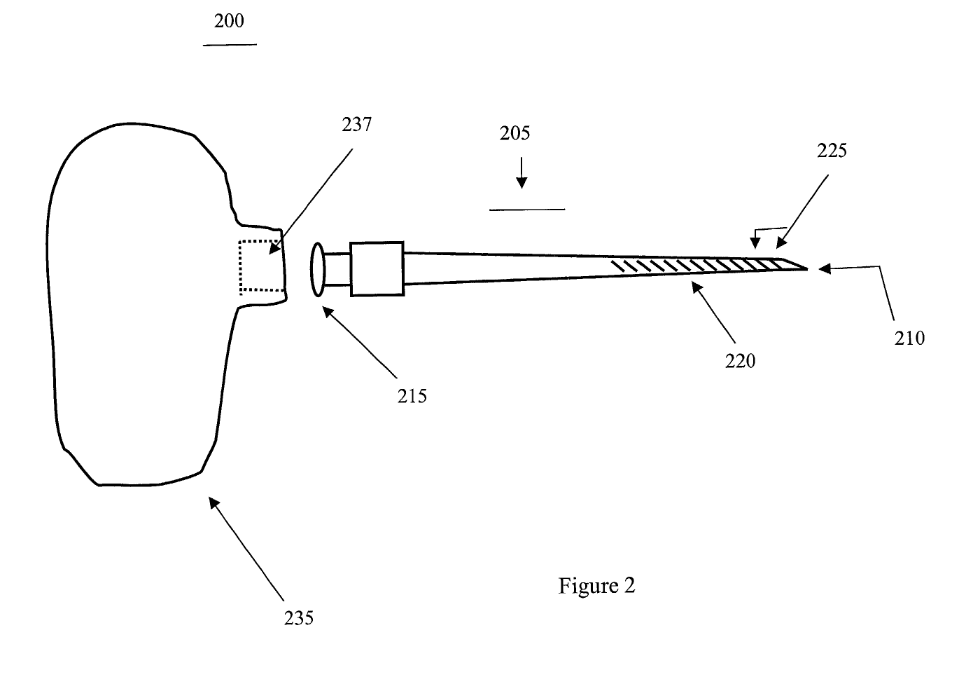

FIG. 2 shows a further embodiment 200 of the subject invention which comprises a catheter 205 for injecting biomaterial into a site of need. The catheter 205 comprises a sharp trocar end 210 and opposite to this end is a luer-lok end 215 for attaching a syringe or other device that contains biomaterial to be injected. Proximate to the trocar end 210 are threads 220 disposed on the catheter 205. Also shown are holes 225 provided proximate to the trocar end to allow for delivery of the selected biomaterial. Preferably, the holes are position ninety degrees relative to each other. To facilitate manipulation of the catheter 205, a handle 235 is shown comprising a socket 237 for engaging the luer-lok end 215.

The resulting repair using a bone paste composition leads to a mass of mineralized tissue that is vascularized. The vascular nature of the tissue ensures that it is less likely to become infected. It will also be easier to re-operate if it should become necessary. Finally, the materials used herein is not as radiodense as bone cement, and thus will not obstruct the underlying structures or cause x-ray artifacts. A non-degradable hydroxyapatite composition provides the additional advantage of having a long residence time in the vertebral body with proximal healthy tissues induced by the osteoinductive factors in the subject pastes. This mass is stable and not as subject to degradation by the osteoporotic patient. The chances for long-term success of an implant of this sort are, therefore, higher than those known in the art.

It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method of treating osteoporotic degradation of a bone comprising the steps of:
engaging a catheter device into said bone, said catheter device being elongated and comprising a connector disposed thereon for connecting an injection device containing a filler; and
injecting said filler into said bone.

2. The method of claim 1 wherein said catheter device comprises a first end having threads disposed thereon and a second end having said connector disposed thereon, wherein said threads are suitable for engaging said catheter to said bone.

3. The method of claim 1 wherein said connector is a luer-lok fitting.

4. The method of claim 1 wherein said bone is a vertebra, clavicle, femur, scapula, or humerus.

5. The method of claim 4 wherein said bone is a vertebra.

6. The method of claim 1 wherein said method is minimally invasive.

7. The method of claim 1 wherein said injection device is a syringe.

8. The method of claim 1 wherein said filler comprises corticocancellous chips (CCC), ground bone powder, non-degradable hydroxyapatite, degradable hydroxyapatite, non-degradable bioglass, degradable bioglass, osteogenic paste, chondrogenic paste, carrier associated Growth Factors, carrier associated mineralized particles, morsellized skin or other tissue, Fibrin powder, Fibrin/plasminogen glue, Demineralized Bone Matrix (DBM)/glycerol, DBM/pleuronic F127, DBM/CCC/F127, polyesters, polyhydroxy, compounds, polyvinyl compounds, polyamino compounds, polycarbonate compounds, or combinations thereof.

9. The method of claim 2 wherein said threads are self-tapping.

10. A kit for repairing osteoporotic bone comprising:
a catheter device, said catheter being elongated and comprising a first end having threads disposed thereon and a second end having an attachment means disposed thereon; and
a syringe containing a filler.

11. The kit of claim 8 wherein said filler comprises corticocancellous chips (CCC), ground bone powder, non-degradable hydroxyapatite, degradable hydroxyapatite, non-degradable bioactive glass, degradable bioactive glass, osteogenic paste, chondrogenic paste, carrier associated Growth Factors, carrier associated mineralized particles, morsellized skin or other tissue, Fibrin powder, Fibrin/plasminogen glue, Demineralized Bone Matrix (DBM)/glycerol, DBM/pleuronic F127, DBM/CCC/F127, polyesters, polyhydroxy, compounds, polyvinyl compounds, polyamino compounds, polycarbonate compounds, or a combinations thereof.

12. The kit of claim 9 wherein said filler comprises CCC of a size from about 100 to about 1000 microns, ground bone powder having a particle size of about 100 to about 1000 microns, non-degradable or degradable hydroxyapatite, bioactive glass, or combinations thereof.

13. The kit of claim 10 wherein the size of said CCC or said particle size is from about 250 to about 950 microns.

14. The kit of claim 11 wherein the size of said CCC or said particle size is from about 350 to about 900 microns.

15. The kit of claim 12 wherein the size of said CCC or said particle size is from about 500 to about 850 microns.

16. A device for injecting materials into bone comprising a threaded catheter and an internal removable trocar.
